(19) United States
(12) Patent Application Publication
(10) Pub. No.: US 2014/0005475 A1
(43) Pub. Date: Jan. 2, 2014

(54) Image Tracking System and Image Tracking Method Thereof

(71) Applicant: NATIONAL CHIAO TUNG UNIVERSITY, Hsinchu City (TW)

(72) Inventors: Kai-Tai Song, Hsinchu City (TW); Chun-Ju Chen, Taichung City (TW)

(73) Assignee: National Chiao Tung University, Hsinchu City (TW)

(21) Appl. No.: 13/677,057

(22) Filed: Nov. 14, 2012

(30) Foreign Application Priority Data
Jun. 27, 2012 (TW) 101123100

(51) Int. Cl. A61B 1/313; A61B 1/00; A61B 1/045

(52) U.S. Cl. CPC A61B 1/3132; A61B 1/045; A61B 1/00009; A61B 1/00006
USPC 600/109

(57) ABSTRACT

An image tracking system and an image tracking method. The image tracking system includes image capture module, detection module and processing module. The image capture module captures real-time image. The detection module analyzes the real-time image, and detects whether positions of a plurality of instruments are disposed in the real-time image. The processing module defines buffer zone in the real-time image, and analyses whether the instruments are disposed in the buffer zone based on the positions of the instruments, and determines whether spacing distance between the instruments is small than preset distance. When the spacing distance is smaller than the preset distance or the instruments are disposed outside the buffer zone, the processing module emits controlling signal to control the image capture module to move to capture position. As a result, the present invention may achieve image tracking real time and provide stable image.

### CROSS-REFERENCE TO RELATED APPLICATION

This application claims the benefit of Taiwan Patent Application No. 101123100, filed on Jun. 27, 2012, in the Taiwan Intellectual Property Office, the disclosure of which is incorporated herein in its entirety by reference.

### BACKGROUND OF THE INVENTION

1. Field of the Invention

The present invention relates to an image tracking system and an image tracking method thereof, in particular to the image tracking system and method that use a single endoscopic camera for an independent tracking surgical instrument.

2. Description of the Related Art

In recent years, doctors and patients have increasingly higher willingness to select minimally invasive surgery instead of traditional open surgery. In the past, doctors needed to spare a hand to adjust an endoscope while operating an instrument during a minimally invasive surgery, and some endoscopic systems with a capacity to track surgical instrument were developed to alleviate the doctors' burden in surgical operations.

For example, U.S. Pat. No. 5,820,545 disclosed the use of two camera devices to capture an image to identify a position of a color coded marking at a front end of a surgical instrument, and the depth of an image is derived according to an aberration of the image to maintain the distance between an endoscope and the surgical instrument, so that the surgical instrument may be controlled at an image center. However, this patented technology may not control the distance between the camera and a target by using a single-lens camera or control the camera to be closer to the instrument for a precise operation. U.S. Pat. No. 5,836,869 disclosed an image tracking endoscope system that uses a switch to control the magnification, focus and view field of a camera to obtain a better operating view. However, this patented technology requires users to control and operate the switch manually, but the system may not track an instrument independently.

In a minimally invasive surgery, the position of an instrument is the position for an operating surgeon to perform the operation. In general, an assistant surgeon assists the operating surgeon to control the endoscope, and now, a robot is generally used for operating the endoscope and providing images by a tracking instrument. However, the mechanical tracking may move the camera too much and thus it incurs difficulty and visual burden for the operating surgeon to perform the operation.

As some operations may require two or more surgical instruments, the prior art may not track three or more instrument positions simultaneously. Therefore, it is urgent and important for related designers and manufacturer to develop an image tracking system and an image tracking method to provide stable images and track a plurality of surgical instruments, wherein the distance between surgical instruments is used to control the endoscopic camera device to perform a 3D image tracking.

### SUMMARY OF THE INVENTION

In view of the aforementioned problems of the prior art, one of the objectives of the present invention is to provide an image tracking system and an image tracking method thereof to track an image stably, and the system and method are applicable for complicated surgeries that require a plurality of surgical instruments, and three-dimensional movements of the camera device may be controlled to overcome the aforementioned problems of the prior art. In the meantime, the present invention uses an image provided by an endoscope controlling robot to stabilize a surgical screen to achieve an effective tracking, wherein an appropriate buffer zone is selected at the center of the image to avoid too much unnecessary movements of the camera in order to provide a stable screen quality.

To achieve the foregoing objective, the present invention provides an image tracking system comprising an image capture module, a detection module and a processing module. The image capture module captures a real-time image. The detection module analyzes the real-time image and detects whether the positions of a plurality of instruments are disposed in the real-time image. The processing module is electrically coupled to the detection module, and a buffer zone is defined in the real-time image, and an analysis is performed to determine whether the instruments are disposed in the buffer zone of the real-time image according to the positions of the plurality of instruments. When the instruments are disposed in the buffer zone of the real-time image, the processing module calculates a spacing distance between the instruments and determines whether the spacing distance between the instruments is smaller than a first preset distance. If the spacing distance is smaller than the first preset distance, then the processing module will emit a controlling signal to control the image capture module to move to a capture position.

Wherein, the detection module analyzes the positions of the plurality of instruments according to a color ring marked on the plurality of instruments.

Wherein, the detection module analyzes a center coordinate of the color ring to obtain the center coordinate to identify the positions of the plurality of instruments.

Wherein, the detection module analyzes a color area of the color ring, and if the color area is smaller than a color threshold value, the detection module determines that the instruments do not exist in the real-time image.

Wherein, the processing module calculates an axial movement parameter of the image capture module and emits the controlling signal to control the image capture module to move towards the instruments according to the axial movement parameter, if the spacing distance is smaller than the first preset distance.

Wherein, the processing module controls the axis of the image capture module to be locked and to remain still, if the detection module detects one of the instruments or the instruments are not disposed in the real-time image.

Wherein, the processing module releases the locking of axis of the image capture module, if the detection module detects that the instruments are disposed in the real-time image.

Wherein, the detection module detects whether the positions of the plurality of instruments still remain in the real-time image if the instruments are disposed in the buffer zone of the real-time image and the spacing distance between the instruments is greater than the first preset distance.

Wherein, the processing module determines whether the spacing distance between the instruments is greater than a second preset distance if the instruments are disposed in the real-time image and inside the buffer zone; if yes, then the processing module calculates an axial movement parameter of the image capture module and emits the controlling signal according to the axial movement parameter to control the image capture module to move away from the instruments.

Wherein, the processing module calculates a planar movement parameter of the image capture module to control the image capture module to approach the instruments so as to a real-time image of the instruments again into the buffer zone, if the instruments are disposed outside the buffer zone.

To achieve the aforementioned objective, the present invention further provides an image tracking method, and the image tracking method is applicable in an image tracking system, and the image tracking system comprises an image capture module, a detection module and a processing module, and the image tracking method comprises the steps of: capturing a real-time image by the image capture module; analyzing the real-time image by the detection module; detecting whether positions of a plurality of instruments are disposed in the real-time image by the detection module; defining a buffer zone in the real-time image by the processing module; using the processing module to analyze whether the instruments are disposed in the buffer zone of the real-time image according to the positions of the instruments; using the processing module to calculate a spacing distance between the instruments and determine whether the spacing distance between the instruments is smaller than a first preset distance if the instruments are disposed in the buffer zone of the real-time image; and using the processing module to emit a controlling signal to control the image capture module to move to a capture position, if the spacing distance is greater than a second preset distance.

In summation, the image tracking system and method of the present invention have one or more of the following advantages:

(1) The image tracking system and method of the present invention may prevent unstable screen caused by a quick movement of the surgical instrument which will affect a surgeon's surgical operation.

(2) The image tracking system and method of the present invention may provide images to medical professionals to operate an endoscope and a surgical instrument stably during a surgical operation.

(3) The image tracking system and method of the present invention may track a plurality of surgical instruments simultaneously and controls an endoscopic camera device to perform 3D image tracking according to the distance between the surgical instruments.

The aforementioned and other objectives, technical characteristics and advantages of the present invention will become apparent with the detailed description of preferred embodiments accompanied with the illustration of related drawings as follows.

### BRIEF DESCRIPTION OF THE DRAWINGS

FIG. 1 is a block diagram of an image tracking system of the present invention;

FIG. 2 is a first schematic view of an image tracking system of the present invention;

FIG. 3 is a second schematic view of an image tracking system of the present invention;

FIG. 4 is a first schematic view of an image tracking method of the present invention; and

FIG. 5 is a second schematic view of an image tracking method of the present invention.

### DESCRIPTION OF THE PREFERRED EMBODIMENTS

The following related drawings are provided for the purpose of illustrating an image tracking system and an image tracking method thereof in accordance with the present invention, and it is noteworthy that same numerals used in the following preferred embodiments represent respective same elements respectively.

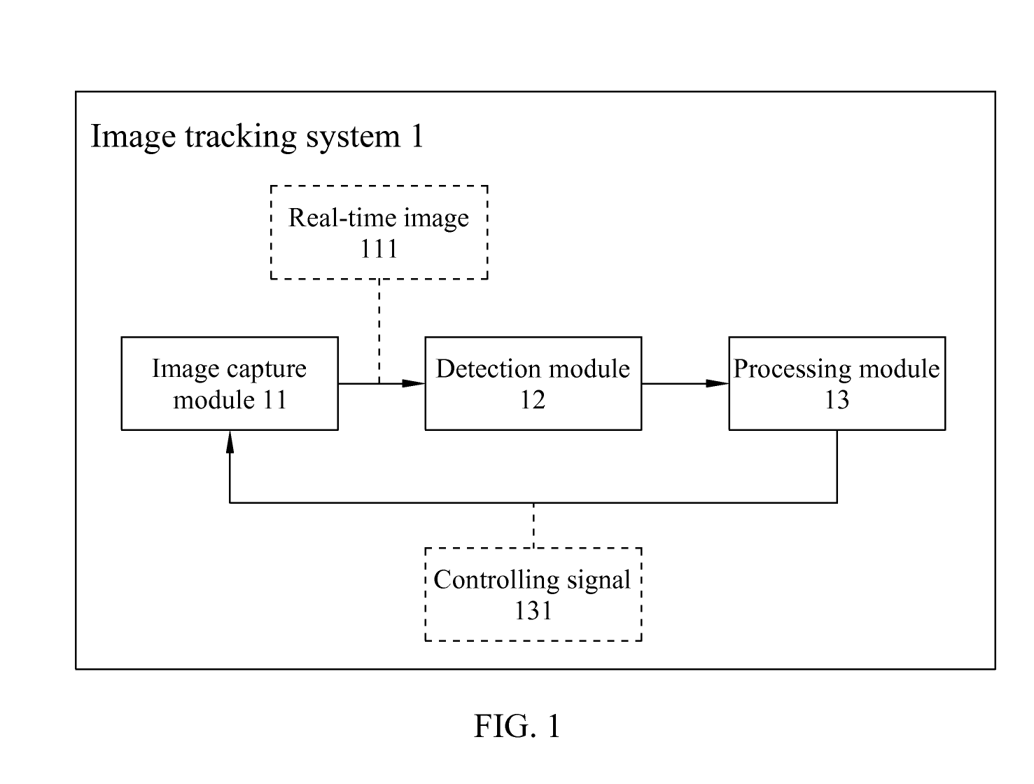

With reference to FIG. 1 for a block diagram of an image tracking system of the present invention, the image tracking system 1 comprises an image capture module 11, a detection module 12 and a processing module 13. Wherein, the image capture module 11 is provided for capturing a real-time image 111, and the image capture module 11 is a light sensing component such as a complementary metal oxide semiconductor (CMOS), a charge coupled device (CCD), or an endoscopic lens. The detection module 12 is provided for analyzing the real-time image 111 and detecting positions of the plurality of instruments to determine whether the instruments are disposed in the real-time image 111. The processing module 13 is electrically coupled to the detection module 12, and the processing module 13 is a central processing unit (CPU) or a micro-processing unit. The processing module 13 includes a buffer zone defined in the real-time image 111 and the processing module 13 is provided for analyzing whether the instruments are disposed in the buffer zone of the real-time image 111 according to the positions of the instruments or calculating a spacing distance between the plurality of instruments and determining whether the spacing distance between the instruments is smaller than a first preset distance or greater than a second preset distance, and the processing module 13 emits a controlling signal 131 to control the image capture module 11 to move to a capture position.

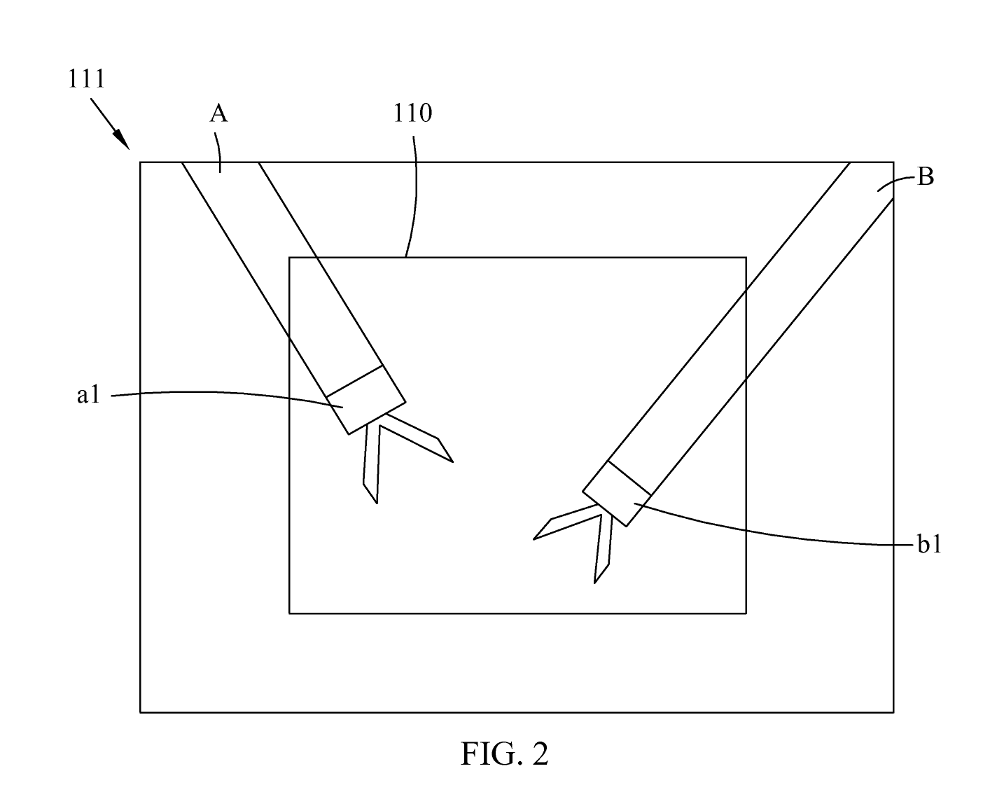

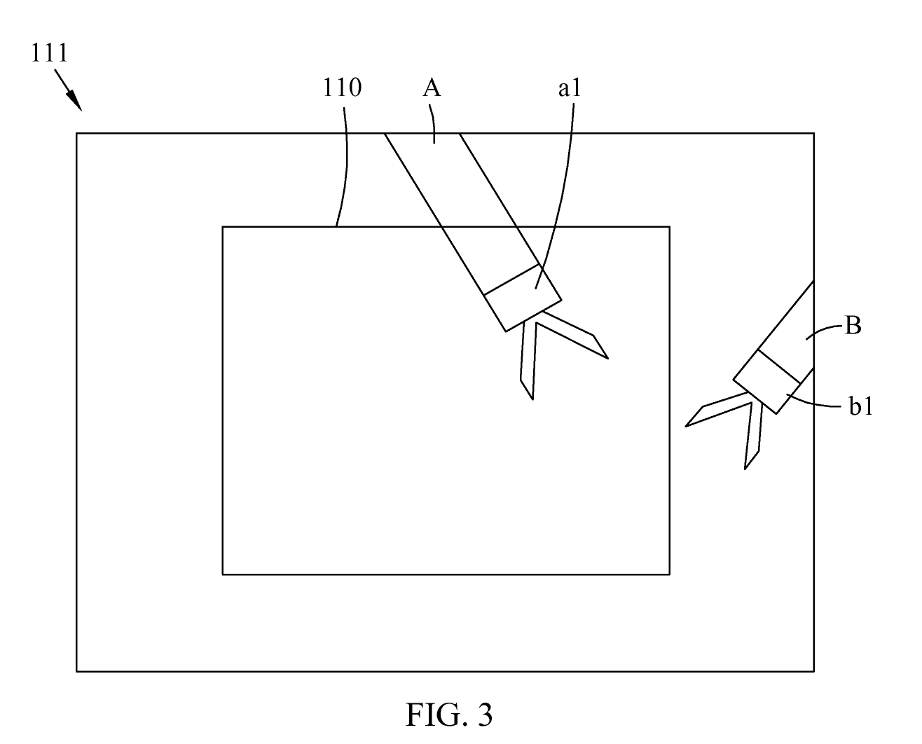

With reference to FIGS. 2 and 3 for the first and second schematic views of an image tracking system in accordance with a preferred embodiment of the present invention. In this preferred embodiment, two instruments (instrument A and instrument B) are used as examples for illustrating the surgical instrument, but the actual quantity of instruments used is not limited. In FIG. 1, the processing module 13 of the present invention defines a buffer zone 110 in the real-time image 111 and marks a color ring a1 on the instrument A and a color ring b1 on the instrument B, so that the detection module 12 may detect positions of the instruments A and instrument B quickly. In other words, different color rings are marked on the instruments of the present invention, and the detection module 12 may distinguish different instruments according to the color ring marked on the plurality of instruments, and further analyze a center coordinate on the color ring, so that the detection module 12 may obtain the positions of the plurality of instruments. In addition, the detection module 12 of the present invention analyzes a color area of the color ring and determines whether the color area is smaller than a color threshold value. If the color area is smaller than the color threshold value, the instrument is determined to be not in the real-time image 111. If the color area is greater than or equal to the color threshold value, the instrument is determined to be in the real-time image 111.

If the detection module detects that one of the instrument A and the instrument B or both instrument A and instrument B are not in the real-time image 111, the processing module 13 controls the axis of the image capture module 11 to be locked and to remain still. If the detection module 12 detects that both instrument A and instrument B are in the real-time image 111, the processing module 13 releases the locking of the axis of the image capture module 11. The processing module 13 defines a buffer zone 110 in the real-time image 111 in advance, and analyzes whether the instruments are in the buffer zone 110 of the real-time image 111 according to the positions of the plurality of instruments. If the instruments are in the buffer zone 110 of the real-time image 111, the processing module 13 calculates a spacing distance between the instruments and determines whether the spacing distance between the instruments is smaller than a first preset distance.

Now, the first preset distance is a threshold value for determining whether the distance between the instruments in the screen is too close, so that the distance between the instruments is used to determine whether the distance between the instruments is too close. If the spacing distance is smaller than the first preset distance, the processing module 13 may calculate an axial movement parameter of the image capture module 11 and emit a controlling signal 131 according to the axial movement parameter to control the image capture module 11 to move towards the plurality of instruments. However, if the spacing distance between the plurality of instruments is greater than the first preset distance, the image capture module 11 will stop moving, and the detection module 12 will continue detecting the real-time image 111.

If the spacing distance between the plurality of instruments is greater than the second preset distance, the processing module 13 is used to determine that the distance between the plurality of instruments is too far, the processing module 13 calculates the axial movement parameter of the image capture module 11 and emit a controlling signal 131 according to the axial movement parameter to control the image capture module 11 to away from the plurality of instruments. If the spacing distance between the plurality of instruments is smaller than the second preset distance, the image capture module 11 will stop moving, and the detection module 12 will continue detecting the real-time image 111.

As show in FIG. 3, although both instrument A and instrument B falls within the range of the real-time image 111, only the instrument A is situated in the buffer zone 110, the instrument B has been disposed outside the buffer zone 110, the processing module 13 calculates a planar movement parameter of the image capture module 11 to control the image capture module 11 to approach the instrument B outside the buffer zone 110 according the instrument B outside the buffer zone 100 with respect to an offset of a boundary of the buffer zone 110. Even if the real-time image 111 is as shown FIG. 3 at the beginning, the instruments in the screen are not situated at the center of the screen. With the image tracking achieved by the image tracking system 1 of the present invention, the images of the instrument A and the instrument B may be adjusted to the center of the screen (as shown in FIG. 2). The image tracking system 1 of the present invention may finish the track of the instruments by combining with the previous an axial (depth direction) tracking function of the image capture module 11 which is controlled through the first preset distance and the second preset distance.

In FIG. 2, the buffer zone 110 may be defined with different sizes as needed. In general, the larger the buffer zone 110, the broader is the activity range of the surgical instrument. The smaller the activity of the buffer zone 110, the higher is the tracking sensitivity. Therefore, an optimal size of the buffer zone 110 may be defined based on the activity range and the tracking effect of the surgical instrument.

It is noteworthy that the image capture module 11 of the present invention may be an endoscopic lens, so that the invention may be applied for two-dimensional movements as well as three-dimensional movements (including the axial movement). Therefore, the movement parameters include an axial movement parameter and a planar movement parameter.

Even though the concept of the image tracking method of the image tracking system 1 of the present invention has been described in the section of the image tracking system 1, the following flow chart is provided for describing the concept more clearly.

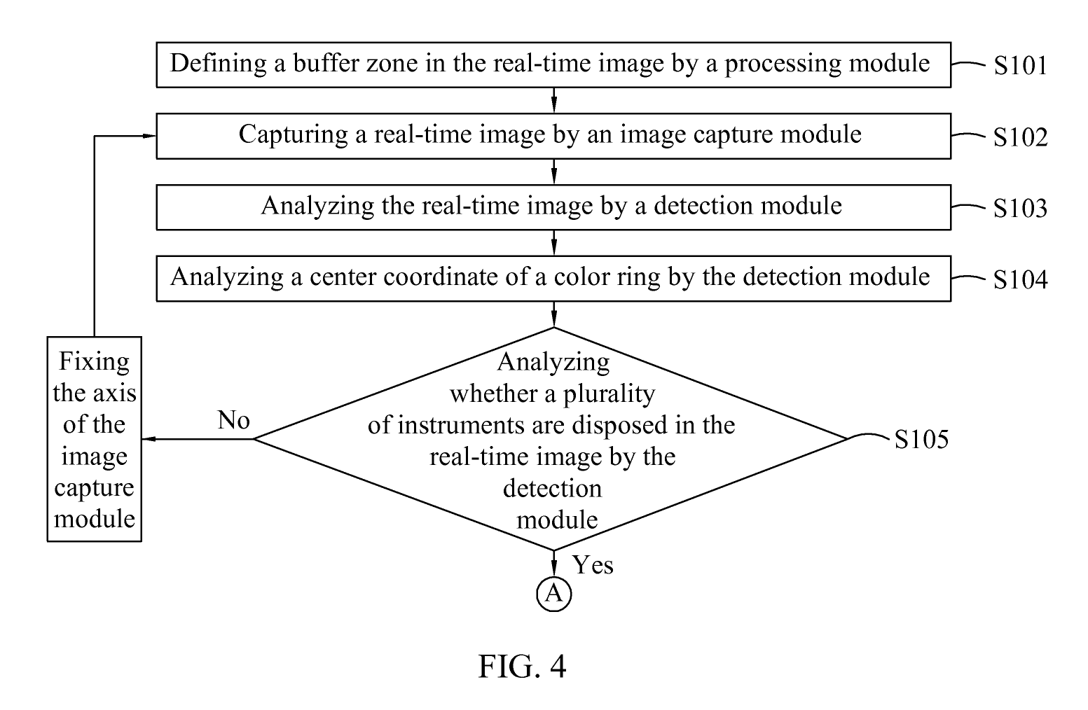

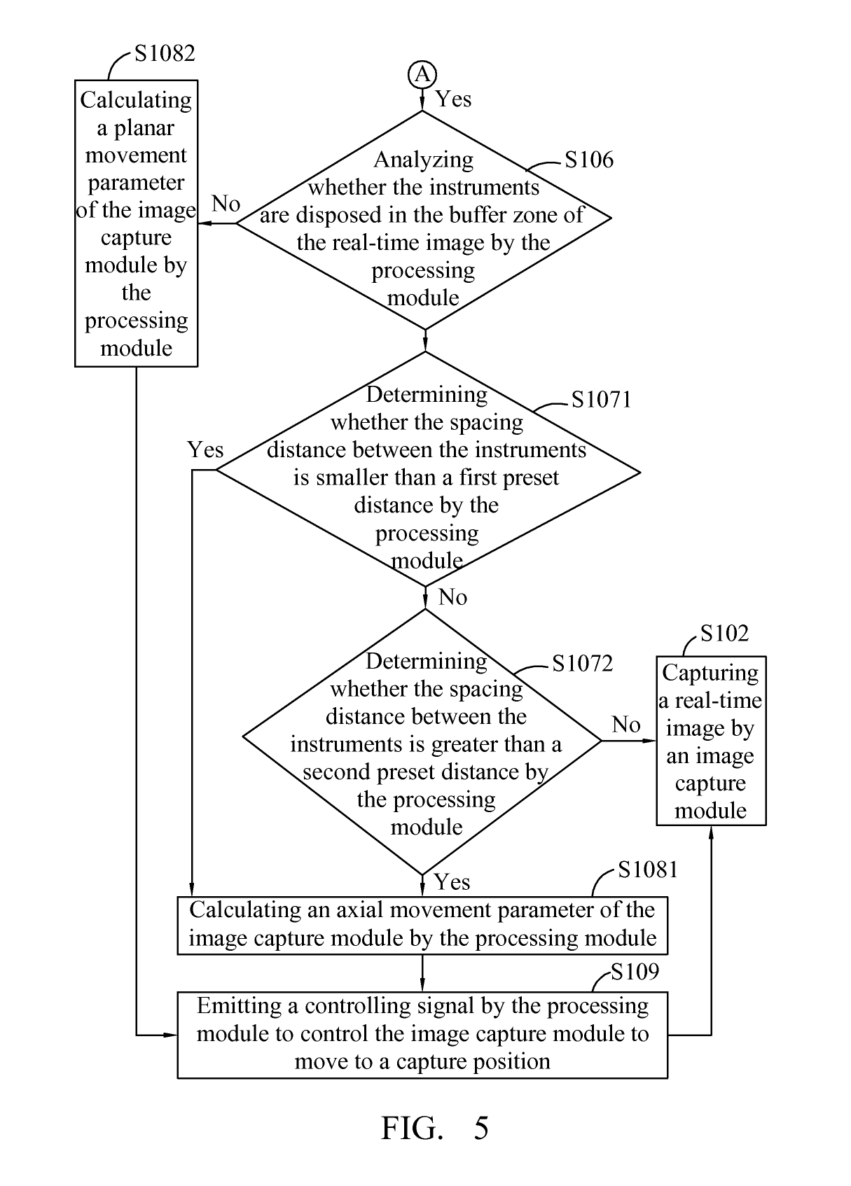

With reference to FIGS. 4 and 5 for the first and second flow charts of an image tracking method of the present invention, the image tracking method is applicable in an image tracking system as described above, and thus will not be repeated. The image tracking method comprises the following steps:

S101: Defining a buffer zone in a real-time image by a processing module.

S102: Capturing the real-time image by an image capture module.

S103: Analyzing the real-time image by a detection module.

S104: Analyzing a center coordinate of a color ring by the detection module.

S105: Analyzing whether a plurality of instruments are disposed in the real-time image by the detection module. If yes, then go to S106, or fixing the axis of the image capture module and else go to S102.

S106: Analyzing whether the instruments are disposed in the buffer zone of the real-time image by the processing module. If yes, then go to S1071, or else go to S 1082.

S1071: Determining whether the spacing distance between the instruments is smaller than a first preset distance by the processing module. If yes, then go to S1081, or else go to S1072.

S1072: Determining whether the spacing distance between the instruments is greater than a second preset distance by the processing module. If yes, then go to S1081, or else go to S102.

S1081: Calculating an axial movement parameter of the image capture module by the processing module.

S1082: Calculating a planar movement parameter of the image capture module by the processing module.

S109: Emitting a controlling signal by the processing module to control the image capture module to move to a capture position.

The detailed description and implementation of the image tracking method of the present invention have been described in the section of the image tracking system, and thus will not be repeated.

While the invention has been described by means of specific embodiments, numerous modifications and variations could be made thereto by those skilled in the art without departing from the scope and spirit of the invention set forth in the claims.

What is claimed is:

1. An image tracking system, comprising:
an image capture module, capturing a real-time image;
a detection module, analyzing the real-time image, and detecting positions of a plurality of instruments to determine whether the positions of the plurality of instruments are disposed in the real-time image; and
a processing module, electrically coupled to the detection module, and provided for defining a buffer zone in the real-time image, determining whether the positions of the instruments are disposed in the buffer zone of the real-time image; and calculating a spacing distance between the instruments if the instruments are disposed in the buffer zone of the real-time image, determining whether the spacing distance between the instruments is smaller than a first preset distance; and emitting a controlling signal to control the image capture module to move to a capture position if the spacing distance is smaller than the first preset distance.

2. The image tracking system of claim 1, wherein the detection module analyzes the positions of the plurality of instruments according to a color ring marked on the plurality of instruments.

3. The image tracking system of claim 2, wherein the detection module analyzes a center coordinate of the color ring and obtains the center coordinate to identify the positions of the plurality of instruments.

4. The image tracking system of claim 2, wherein the detection module analyzes a color area of the color ring, and if the color area is smaller than a color threshold value, the detection module determines that the instruments do not exist in the real-time image.

5. The image tracking system of claim 1, wherein the processing module calculates an axial movement parameter of the image capture module and emits the controlling signal to control the image capture module to move towards the instruments according to the axial movement parameter, if the spacing distance is smaller than the first preset distance.

6. The image tracking system of claim 1, wherein the processing module controls the axis of the image capture module to be locked and to remain still, if the detection module detects one of the instruments or the instruments are not disposed in the real-time image.

7. The image tracking system of claim 1, wherein the processing module releases the locking of axis of the image capture module, if the detection module detects that the instruments are disposed in the real-time image.

8. The image tracking system of claim 1, wherein the detection module detects whether the positions of the plurality of instruments still remain in the real-time image if the instruments are disposed in the buffer zone of the real-time image and the spacing distance between the instruments is greater than the first preset distance.

9. The image tracking system of claim 1, wherein the processing module determines whether the spacing distance between the instruments is greater than a second preset distance if the instruments are disposed in the real-time image and inside the buffer zone; if yes, then the processing module calculates an axial movement parameter of the image capture module and emits the controlling signal according to the axial movement parameter to control the image capture module to move away from the instruments.

10. The image tracking system of claim 1, wherein the processing module calculates a planar movement parameter of the image capture module to control the image capture module to approach the instruments so as to a real-time image of the instruments again into the buffer zone according the instruments outside the buffer zone with respect to an offset of a boundary of the buffer zone, if the instruments are disposed outside the buffer zone.

11. An image tracking method, applicable in an image tracking system, and the image tracking system comprising an image capture module, a detection module and a processing module, and the image tracking method comprising the steps of:
capturing a real-time image by the image capture module;
analyzing the real-time image by the detection module;
detecting whether positions of a plurality of instruments are disposed in the real-time image by the detection module;
defining a buffer zone in the real-time image by the processing module;
using the processing module to analyze whether the instruments are disposed in the buffer zone of the real-time image according to the positions of the instruments;
using the processing module to calculate a spacing distance between the instruments and determine whether the spacing distance between the instruments is smaller than a first preset distance if the instruments are disposed in the buffer zone of the real-time image; and
using the processing module to emit a controlling signal to control the image capture module to move to a capture position, if the spacing distance is greater than a second preset distance.

12. The image tracking method of claim 11, further comprising the step of:
analyzing the positions of the plurality of instruments by the detection module according to a color ring marked on the plurality of instruments.

13. The image tracking method of claim 12, further comprising the step of:
analyzing a center coordinate of the color ring by the detection module to obtain the center coordinate to identify the positions of the plurality of instruments.

14. The image tracking method of claim 12, further comprising the steps of:
analyzing a color area of the color ring by the detection module; and determining that the instruments do not exist in the real-time image if the color area is smaller than a color threshold value.

15. The image tracking method of claim 11, further comprising the steps of:
using the processing module to calculate an axial movement parameter of the image capture module, if the spacing distance is smaller than the first preset distance; and emitting the controlling signal by the processing module according to the axial movement parameter to control the image capture module to move towards the instruments.

16. The image tracking method of claim 11, further comprising the step of:
controlling the axis of the image capture module to be locked and to remain still by the processing module, if the detection module detects one of the instruments or the instruments are not disposed in the real-time image.

17. The image tracking method of claim 11, further comprising the step of:
releasing the locking of the axis of the image capture module by the processing module, if the detection module detects that the instruments are disposed in the real-time image.

18. The image tracking method of claim 11, further comprising the step of:
using the detection module to detect whether the positions of the plurality of instruments still remain in the real-time image, if the instruments are disposed in the buffer zone of the real-time image and the spacing distance between the instruments is greater than the first preset distance.

19. The image tracking method of claim 11, further comprising the steps of:
using the processing module to determine whether the spacing distance between the instruments is greater than the second preset distance if the instruments are disposed in the real-time image and inside the buffer zone; and if yes, using the processing module to calculate an axial movement parameter of the image capture module; and using the processing module to emit the controlling signal to control the image capture module to move away from the instruments according to the axial movement parameter.

20. The image tracking method of claim 11, further comprising the step of:
using the processing module to calculate a planar movement parameter of the image capture module to control the image capture module to approach the instruments so as to a real-time image of the instruments again into the buffer zone according the instruments outside the buffer zone with respect to an offset of a boundary of the buffer zone, if the instruments are disposed outside the buffer zone.
